(19) United States
(12) Patent Application Publication
(10) Pub. No.: US 2024/0325400 A1
(43) Pub. Date: Oct. 3, 2024

(54) Composition Comprising Skin-Specific T Cell Inhibitor as Active Ingredient for Prevention or Treatment of Atopic Dermatitis

(71) Applicant: INDUSTRY-ACADEMIC COOPERATION FOUNDATION, YONSEI UNIVERSITY, Seoul (KR)

(72) Inventors: Chang Ook PARK, Seoul (KR); Yu Ri KIM, Seoul (KR); Su Min KIM, Seoul (KR); Hye Li KIM, Goyang-si (KR); Kelun ZHANG, Seoul (KR); Hyun Soo KIM, Seoul (KR)

(21) Appl. No.: 18/707,206

(22) PCT Filed: Nov. 4, 2022
(86) PCT No.: PCT/KR2022/017214

§ 371 Date: May 3, 2024

(30) Foreign Application Priority Data
Nov. 5, 2021 (KR) 10-2021-0151317

(51) Int. Cl. A61K 31/52; A61P 17/00; A61P 29/00; G01N 33/50

(52) U.S. Cl. CPC A61K 31/52; A61P 17/00; A61P 29/00; G01N 33/5023; G01N 33/505

(57) ABSTRACT

The present invention relates to a composition for prevention or treatment of inflammatory or autoimmune skin diseases. The composition of the present invention may be useful as a fundamental therapeutic composition that is able to efficiently ameliorate skin tissue damage caused by inflammation while having a minimized impact on systemic immune activity by specifically inhibiting the activity of skin-specific T cells. In addition, the composition of the present invention may be efficiently delivered to the stratum corneum of the skin, so that it is able to exert a lesion-specific and intensive immune control effect as a composition for topical application to the skin. Moreover, it shows little toxicity to normal skin tissue, and thus is suitable even for long-term administration for the treatment of atopic dermatitis, a chronic disease. The present invention also provides, based on the newly identified correlation between skin-specific T cells and PIK3CD protein, a screening method capable of quickly and highly reliably identifying promising therapeutic candidates that are able to alleviate various autoimmune and inflammatory damages occurring in skin tissue.

### CROSS-REFERENCE TO RELATED APPLICATIONS

This application is a U.S. National Stage Entry of International Patent Application no. PCT/KR2022/017214, filed Nov. 4, 2022, which claims the benefit of priority of Korean Patent Application no. 10-2021-0151317, filed Nov. 5, 2021.

### TECHNICAL FIELD

The present invention relates to a method of treating inflammatory and autoimmune skin diseases, including atopic dermatitis, using a small-molecule compound that selectively inhibits skin-specific T cells.

### BACKGROUND ART

Atopic dermatitis is an incurable skin disease that has a complex pathogenesis and chronically relapses, and the primary cause thereof is the hypersensitivity of the immune system to external stimuli. Currently, steroids or immunosuppressants such as cyclosporine, azathioprine, and mycophenolate mofetil (MMF) are mainly used to treat atopic dermatitis, but these therapeutic agents have temporary effects or are not suitable for long-term administration. Thereamong, topical steroids have a broad spectrum of effects and are drugs that exhibit the most significant effect as they target various immune molecules in addition to type 2 inflammation. However, these topical steroids cause serious problems when used continuously. For example, in the case of infants and young children having a large body surface area in relation to their body weight, the topical steroids may be absorbed throughout the body, which may cause side effects such as adrenal suppression, and in the case of elderly people with thin skin, there is a high risk of side effects due to excessive absorption of the steroid drugs.

A topical calcineurin inhibitor (TCI), developed to overcome the side effects of steroid drugs, has been shown to have an anti-inflammatory effect similar to steroid drugs and improve the quality of life of patients by reducing itching. However, because the topical calcineurin inhibitor has a molecular weight of over 800 Da, its efficacy is limited due to its low absorption rate in thick skin lesions, and in cases of moderate or higher severity, the topical calcineurin inhibitor has a side effect of lichenification, which is thickening of skin folds.

Meanwhile, one of the important pathogenic mechanisms of atopic dermatitis is that infiltration of T cells into the lesion causes damage to the skin barrier function and impairment of immune system regulation. The biggest problem with existing immunosuppressants is that, even though atopic dermatitis is a tissue-specific disease that occurs on the skin, most attempts to develop immunosuppressants have been made to control immune and inflammatory responses through a systemic approach, especially an approach that targets the blood. Therefore, a new therapeutic approach targeting skin-specific T cells is needed to explore new therapeutic candidates for atopic dermatitis, but studies thereon have not been conducted worldwide.

Throughout the present specification, a number of publications and patent documents are referred to and cited. The disclosure of the cited publications and patent documents is incorporated herein by reference in its entirety to more clearly describe the state of the art to which the present invention pertains and the content of the present invention.

### DISCLOSURE

### Technical Problem

The present inventors have made extensive research efforts to develop a novel small-molecule therapeutic agent that efficiently ameliorates the symptoms of skin lesions by controlling skin tissue-specific inflammatory and immune response in intractable autoimmune skin diseases with complex pathogenic mechanisms, specifically atopic dermatitis. As a result, the present inventors have found that a quinazolinone derivative compound of Formula (1) below significantly ameliorates skin lesions while having a minimized impact on systemic immune activity by significantly reducing serum total IgE and eosinophil count and remarkably inhibiting skin-specific T cells while showing little toxicity to normal skin tissue, thereby completing the present invention.

Therefore, an object of the present invention is to provide a composition for preventing or treating inflammatory or autoimmune skin disease.

Another object of the present invention is to provide a method for screening a composition for inhibiting skin-specific T cells.

Other objects and advantages of the present invention will be more apparent from the following detailed description, the appended claims, and the accompanying drawings.

### Technical Solution

According to one aspect of the present invention, the present invention provides a composition for preventing or treating inflammatory or autoimmune skin disease, comprising a compound of the following Formula 1 or a pharmaceutically acceptable salt thereof as an active ingredient:

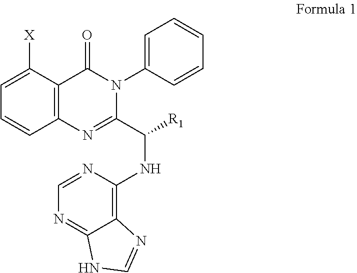

In Formula 1 above, X is halogen, and R₁ is C₁-C₃ alkyl.

The present inventors have made extensive research efforts to develop a novel small-molecule therapeutic agent that efficiently ameliorates the symptoms of skin lesions by controlling skin tissue-specific inflammatory and immune response in intractable autoimmune skin diseases with complex pathogenic mechanisms, specifically atopic dermatitis. As a result, the present inventors have found that the quinazolinone derivative compound of Formula (1) significantly ameliorates skin lesions while having a minimized impact on systemic immune activity by significantly reducing serum total IgE and eosinophil count and remarkably inhibiting skin-specific T cells while showing little toxicity to normal skin tissue.

In the present specification, the term “alkyl” refers to a straight-chain or branched saturated hydrocarbon group, and the term “C₁-C₃ alkyl” refers to an alkyl group having an alkyl unit having 1 to 3 carbon atoms, and when the C₁-C₃ alkyl is substituted, the carbon atom number of the substituent is not included.

In the present specification, the term “halogen” refers to the halogen group elements and includes, for example, fluoro, chloro, bromo, and iodo.

According to a specific embodiment of the present invention, X in Formula 1 is F.

According to a specific embodiment of the present invention, R₁ in Formula 1 is C₂ alkyl.

The compound of Formula 1, wherein X is F, R₁ is isopropyl, and R₂ is C₂ alkyl (ethyl), is idelalisib (CAL-101, C₂₂H₁₈FN₇O). Idelalisib is a small-molecule compound that was developed as an inhibitor of phosphoinositide 3-kinase and is currently used as a second-line therapeutic agent for chronic lymphocytic leukemia (CLL). Idelalisib is a small-molecule compound approved as a therapeutic agent for blood cancers such as B-cell non-Hodgkin lymphoma and relapsed small lymphocytic lymphoma. There have been no reports of the therapeutic effect of idelalisib on autoimmune skin diseases, including atopic dermatitis, as well as its ability to regulate the activity of skin-specific T cells.

In the present specification, the term “inflammatory or autoimmune skin disease” refers to a disease in which skin tissue is damaged and the inherent biological functions of skin tissue, including barrier function, are reduced, due to excessive or unwanted immune response or inflammation caused thereby. As shown in the examples described below, the composition of the present invention is able to exert a lesion-specific and intensive immune control effect compared to common immunosuppressants, which cause systemic immune depression, by significantly ameliorating skin lesions and significantly reducing serum total IgE and eosinophil count, as well as reducing the number and activity of skin-specific T cells present in skin tissues such as the epidermis and dermis, in an atopic dermatitis animal model.

According to a specific embodiment of the present invention, the inflammatory or autoimmune skin disease to be prevented or treated by the composition of the present invention is selected from the group consisting of atopic dermatitis, eczema, erythema multiforme, erythema nodosum, and pyoderma gangrenosum. More specifically, the inflammatory or autoimmune skin disease is atopic dermatitis.

In the present specification, the term “prevention” means inhibiting the occurrence of a disorder or a disease in a subject who has never been diagnosed as having the disorder or disease, but is likely to suffer from such disorder or disease.

In the present specification, the term “treatment” means (a) inhibiting the progress of a disorder, disease or symptom: (b) alleviating the disorder, disease or symptom; or (c) eliminating the disorder, disease or symptom. When the composition of the present invention is administered to a subject, it functions to significantly reduce eosinophil count and serum total IgE and inhibit skin-specific T cells present in skin tissue, thereby inhibiting the progress of symptoms caused by excessive immune and inflammatory responses in skin tissue, or eliminating or alleviating the symptoms. Thus, the composition of the present invention may serve as a therapeutic composition for the disease alone, or may be administered in combination with other pharmacological ingredients and applied as a therapeutic aid for the disease. Accordingly, as used in the present specification, the term “treatment” or “therapeutic agent” encompasses the meaning of “treatment aid” or “therapeutic aid agent”.

In the present specification, the term “administration” or “administering” means administering a therapeutically effective amount of the composition of the present invention directly to a subject so that the same amount is formed in the subject's body.

In the present specification, the term “therapeutically effective amount” refers to an amount of the composition containing a pharmacological ingredient sufficient to provide a therapeutic or prophylactic effect to a subject to whom/which the pharmaceutical composition of the present invention is to be administered. Accordingly, the term “therapeutically effective amount” is meant to include a “prophylactically effective amount”.

In the present specification, the term “subject” includes, without limitation, humans, mice, rats, guinea pigs, dogs, cats, horses, cows, pigs, monkeys, chimpanzees, baboons or rhesus monkeys. Specifically, the subject of the present invention is a human.

According to a specific embodiment of the present invention, the composition of the present invention reduces the number or activity of skin-specific T cells.

In the present specification, the term “pharmaceutically acceptable salt” includes a salt derived from a pharmaceutically acceptable inorganic acid, organic acid, or base. Examples of suitable acids include hydrochloric acid, hydrobromic acid, sulfuric acid, nitric acid, perchloric acid, fumaric acid, maleic acid, phosphoric acid, glycolic acid, lactic acid, salicylic acid, succinic acid, toluene-p-sulfonic acid, tartaric acid, acetic acid, trifluroacetic acid, citric acid, methanesulfonic acid, formic acid, benzoic acid, malonic acid, naphthalene-2-sulfonic acid, benzenesulfonic acid, and the like. Salts derived from suitable bases include salts of alkali metals such as sodium, alkaline earth metals such as magnesium, ammonium, and the like.

When the composition of the present invention is prepared as a pharmaceutical composition, the pharmaceutical composition of the present invention comprises a pharmaceutically acceptable carrier. Examples of the pharmaceutically acceptable carrier that is comprised in the pharmaceutical composition of the present invention include, but are not limited to, lactose, dextrose, sucrose, sorbitol, mannitol, starch, gum acacia, calcium phosphate, alginate, gelatin, calcium silicate, microcrystalline cellulose, polyvinylpyrrolidone, cellulose, water, syrup, methyl cellulose, methylhydroxy benzoate, propylhydroxybenzoate, talc, magnesium stearate, and mineral oil, which are commonly used in formulation. The pharmaceutical composition of the present invention may further comprise a lubricant, a wetting agent, a sweetening agent, a flavoring agent, an emulsifying agent, a suspending agent, a preservative, and the like, in addition to the above-described ingredients. Suitable pharmaceutically acceptable carriers and agents are described in detail in Remington's Pharmaceutical Sciences (19^{th} ed., 1995).

The pharmaceutical composition of the present invention may be administered parenterally. Specifically, it may be administered transdermally or subcutaneously, or applied topically to the skin surface. More specifically, the pharmaceutical composition of the present invention is a composition for transdermal administration or topical skin application.

An appropriate dosage of the pharmaceutical composition of the present invention may vary depending on various factors such as formulation method, administration mode, patient's age, weight, sex, pathological condition, diet, administration time, administration route, excretion rate, and reaction sensitivity. A preferred dosage of the pharmaceutical composition of the present invention is within the range of 0.001 to 100 mg/kg for an adult.

The pharmaceutical composition of the present invention may be prepared in a unit dose form or prepared to be contained in a multi-dose container by formulating with a pharmaceutically acceptable carrier and/or excipient, according to a method that may be easily carried out by a person skilled in the art.

According to another aspect of the present invention, the present invention provides a cosmetic composition for alleviating or ameliorating inflammatory or autoimmune skin disease, comprising a compound of the following Formula 1 or a cosmetically acceptable salt thereof as an active ingredient:

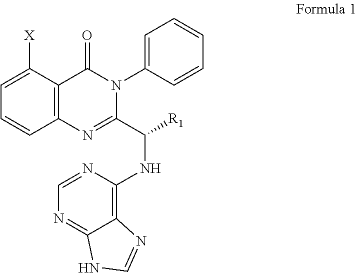

The compound of Formula 1 used in the present invention and the inflammatory or autoimmune skin disease that can be ameliorated or alleviated using the same have already been described in detail above, and thus the description thereof will be omitted to avoid excessive overlapping.

According to a specific embodiment of the present invention, the composition of the present invention reduces the number or activity of skin-specific T cells.

In the present specification, the term “cosmetically acceptable salt” refers to a salt in a form that may be used cosmetically, among salts that are substances in which cations and anions are combined by electrostatic attraction, and refers to the type of salt that can be used cosmetically. Specific examples of the cosmetically acceptable salt include the examples of “pharmaceutically acceptable salts” described above.

Ingredients that are comprised in the cosmetic composition of the present invention include, in addition to the compound of Formula 1 or cosmetically acceptable salt thereof as an active ingredient, ingredients that are commonly used in cosmetic compositions, for example, conventional additives, such as antioxidants, stabilizers, solubilizers, vitamins, pigments and flavors, as well as carriers.

The cosmetic composition of the present invention may be prepared into any formulation which is commonly prepared in the art. For example, it may be formulated into a solution, suspension, emulsion, paste, gel, cream, lotion, powder, soap, surfactant-containing cleanser, oil, powder foundation, emulsion foundation, wax foundation, and spray, without being limited thereto.

If the formulation of the present invention is a paste, cream, or gel, it may comprise, as a carrier ingredient, animal oil, vegetable oil, wax, paraffin, starch, tragacanth, a cellulose derivative, polyethylene glycol, silicone, bentonite, silica, talc, zinc oxide, or the like.

If the formulation of the present invention is a powder or spray formulation, it may comprise, as a carrier ingredient, lactose, talc, silica, aluminum hydroxide, calcium silicate, or polyamide powder. Particularly, if the formulation is a spray formulation, it may further comprise a propellant, such as chlorofluorohydrocarbon, propane/butane, or dimethyl ether.

If the formulation of the present invention is a solution or emulsion, it may comprise, as a carrier ingredient, a solvent, a solubilizing agent, or an emulsifying agent, In this case, examples of the carrier ingredient include water, ethanol, isopropanol, ethyl carbonate, ethyl acetate, benzyl alcohol, benzyl benzoate, propylene glycol, 1,3-butyl glycol oil, glycerol fatty ester, polyethylene glycol, or sorbitan fatty acid ester.

If the formulation of the present invention is a suspension, it may comprise, as a carrier ingredient, a liquid diluent, such as water, ethanol, or propylene glycol, a suspending agent, such as ethoxylated isostearyl alcohol, polyoxyethylene sorbitol ester, or polyoxyethylene sorbitan ester, microcrystalline cellulose, aluminum metahydroxide, bentonite, agar, tragacanth, or the like.

If the formulation of the present invention is a surfactant-containing cleanser, it may comprise, as a carrier ingredient, aliphatic alcohol sulfate, aliphatic alcohol ether sulfate, sulfosuccinic monoester, isethionate, an imidazolium derivative, methyl taurate, sarcosinate, fatty acid amide ether sulfate, alkyl amido betaine, aliphatic alcohol, fatty acid glyceride, fatty acid diethanolamide, vegetable oil, a lanoline derivative, ethoxylated glycerol fatty acid ester, or the like.

According to still another aspect of the present invention, the present invention provides a method for screening a composition for inhibiting skin-specific T cells, comprising steps of:
(a) bringing a candidate substance into contact with a biological sample containing cells expressing PIK3CD (phosphatidylinositol-4,5-bisphosphate 3-kinase catalytic subunit delta); and
(b) measuring the activity or expression level of PIK3CD in the sample,
wherein, if the activity or expression level of PIK3CD decreased, the candidate substance is determined as the composition for inhibiting skin-specific T cells.

In the present invention, the term “biological sample” refers to any sample containing cells expressing PIK3CD, obtained from mammals, including humans, and includes, but is not limited to, tissues, organs, cells, or cell cultures.

According to a specific embodiment of the present invention, the biological sample contains skin tissue or skin tissue-derived cells. The skin tissue-derived cells include, but are not limited to, keratinocytes and skin fibroblasts.

The term “candidate substance” used while referring to the screening method of the present invention refers to an unknown substance that is added to a sample containing cells expressing PIK3CD and is used in screening to examine whether or not it affects the activity or expression level of PIK3CD at the gene or protein level. Examples of the test substance (candidate substance) include, but are not limited to, compounds, nucleotides, peptides, and natural extracts. The step of measuring the expression level or activity of PIK3CD in the biological sample treated with the test substance may be performed by various expression level and activity measurement methods for genes and proteins known in the art. As a result of the measurement, when the expression level or activity of PIK3CD decreased, the test substance may be determined as an inhibitor that reduces the number and/or activity of skin-specific T cells present in skin tissue.

In the present specification, the term “decrease in activity or expression level” means that the in vivo unique function or expression level of PIK3CD decreases to the extent that the activation of skin-specific T cells by PIK3CD as identified by the present inventors is significantly inhibited and the inflammatory damage in skin lesions is ameliorated to measurable levels. A decrease in the activity includes not only a simple decrease in function but also an ultimate decrease in the activity due to a decrease in stability. Specifically, the term “decrease in activity or expression level” may mean a state in which the activity or expression level decreased by at least 20%, more specifically at least 40%, even more specifically at least 60%, compared to that in a control group.

According to yet another aspect of the present invention, the present invention provides a method for preventing, treating or ameliorating inflammatory or autoimmune skin disease, comprising a step of administering to a subject a compound of the following Formula 1 or a pharmaceutically acceptable salt thereof:

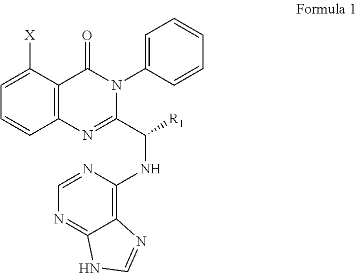

The compound of Formula 1 used in the present invention and the inflammatory or autoimmune skin disease that can be prevented, treated or ameliorated using the same have already been described in detail above, and thus the description thereof will be omitted to avoid excessive overlapping.

### Advantageous Effects

(a) The present invention provides a composition for preventing or treating inflammatory or autoimmune skin disease.

(b) The composition of the present invention may be useful as a fundamental therapeutic composition that is able to efficiently ameliorate skin tissue damage caused by inflammation while having a minimized impact on systemic immune activity by specifically inhibiting the activity of skin-specific T cells.

(c) In addition, the composition of the present invention may be efficiently delivered to the stratum corneum of the skin, so that it is able to exert a lesion-specific and intensive immune control effect as a composition for topical application to the skin. Moreover, it shows little toxicity to normal skin tissue, and thus is suitable even for long-term administration for the treatment of atopic dermatitis, a chronic disease.

(d) The present invention also provides, based on the newly identified correlation between skin-specific T cells and PIK3CD protein, a screening method capable of quickly and highly reliably identifying promising therapeutic candidates that are able to alleviate various autoimmune and inflammatory damages occurring in skin tissue.

### BRIEF DESCRIPTION OF DRAWINGS

FIG. 1 shows a schematic diagram of the PI3K signaling cascade (FIG. 1A) and a heat map for proteins whose expression in skin-specific T cells present in skin tissue of atopic dermatitis increased, among signaling proteins expressed in the PI3K signaling pathway (FIG. 1B). FIG. 1C] depicts bar graphs showing the change in gene expression of PI3KCD shown in the heat map of FIG. 1B.

FIG. 2 shows the appearance of an NC/Nga mouse with atopic dermatitis skin lesions induced by treatment with HDM.

FIG. 3 schematically shows the timeline of treatment of an atopic dermatitis mouse model with HDM and idelalisib.

FIG. 4 is a histogram showing the results of MTT assay (CCK-8) performed to evaluate the cytotoxicity of idelalisib to normal skin tissue.

FIG. 5 shows the therapeutic effect of idelalisib in a mouse model with atopic dermatitis induced by HDM.

FIG. 6 is a graph showing the change in scoring atopic dermatitis (SCORAD) index of an atopic dermatitis mouse model by idelalisib treatment.

FIG. 7 is a graph showing the changes in serum total IgE levels of atopic dermatitis mice by idelalisib treatment. Data are expressed as mean±standard deviation (SD). **P<0.01, *P<0.05.

FIG. 8 is a graph showing the changes in serum total DF-specific IgE levels of an atopic dermatitis mouse model by idelalisib treatment. Data are expressed as mean±standard deviation (SD). **P<0.01, *P<0.05.

FIG. 9 shows the results of hematoxylin and eosin (H&E) staining indicating changes in skin tissue (FIG. 9A), changes in epidermal thickness (FIG. 9B), and changes in eosinophil count (FIG. 9C), following administration of idelalisib and dexamethasone after applying house dust mite (HDM) ointment to NC/Nga mice for 4 weeks. Data are expressed as mean±standard deviation (SD). **P<0.01, *P<0.05.

FIG. 10 depicts FACS data (FIG. 10A) and histograms (FIG. 10B) showing the results of comparing T cell fractions in skin-draining lymph nodes of atopic dermatitis mouse model following idelalisib treatment. Data are expressed as mean±standard deviation (SD). **P<0.01, *P<0.05.

FIG. 11 depicts FACS data (FIG. 11A) and histograms (FIG. 11B) showing the results of comparing T cell fractions in spleens of atopic dermatitis mouse model following idelalisib treatment. Data are expressed as mean±standard deviation (SD). **P<0.01, *P<0.05.

FIG. 12 depicts FACS data (FIG. 12A) and histograms (FIG. 12B) showing the results of comparing T cell fractions in skins of atopic dermatitis mouse model following idelalisib treatment. Data are expressed as mean±standard deviation (SD). **P<0.01, *P<0.05.

FIG. 13 depicts FACS data (FIG. 13A) and histograms (FIG. 13B) showing the results of comparing T cell fractions using skin immune cells and skin lesions of atopic dermatitis patients. Data are expressed as mean±standard deviation (SD). **P<0.01, *P<0.05.

### MODE FOR INVENTION

Hereinafter, the present invention will be described in more detail by way of examples. These examples are only for explaining the present invention in more detail, and it will be apparent to those skilled in the art that the scope of the present invention according to the subject matter of the present invention is not limited by these examples.

### EXAMPLES

### Experimental Methods

### Preparation of Animal Models

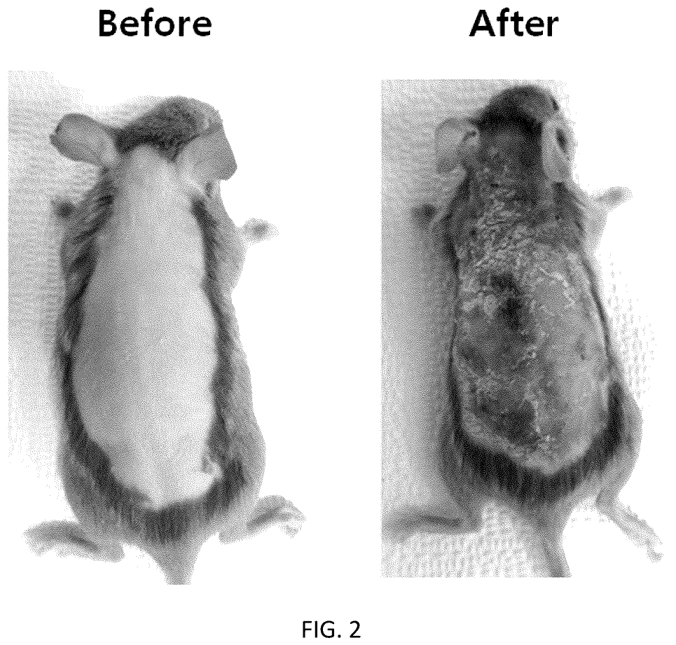

NC/Nga mice showing clinical and histological phenotypes similar to atopic dermatitis were used. The NC/Nga mice were stabilized for 1 week in the laboratory animal room at a temperature of 23±2° C. and a humidity of 55+5% with a 12 hr-light/12-hr dark cycle. To prepare atopic dermatitis animal models, atopic skin lesions were induced by treating mice with either house dust mites (HDM), a representative trigger/aggravator of atopic dermatitis, or a 4% dilution of sodium dodecyl sulfate solution (SIGMA Lot #BCCD5615) (FIG. 2).

After removing the hair on the back of the mouse, dead skin cells were removed with a 4% dilution of sodium dodecyl sulfate (SIGMA Lot #BCCD5615) once every three days, and 100 mg of house dust mite ointment was applied to the back of the mouse. The above procedure was performed for 4 weeks. The house dust mite ointment used was an atopic dermatitis challenging ointment (Biostir, Osaka, Japan), which contains a natural ingredient derived from mite (Dermatophagoides farina). Applying the above ointment could induce house dust mite-specific allergic rhinitis, allergic asthma, or atopic dermatitis, and the ointment was used to induce atopic dermatitis in the present invention.

### Gene Expression Profiles of Skin-Specific T Cells

To identify proteins, whose expression in skin-specific T cells present in skin tissue of atopic dermatitis increased, among signaling proteins expressed in the PI3K signaling pathway, skin tissues were obtained from atopic dermatitis patients (24 to 48 years old) who visited Severance Hospital (Seoul, Korea). To isolate T cells, positive magnetic separation was performed on the cell suspension using human CD3 MicroBeads (Miltenyi Biotec, Bergisch Gladbach, Germany). The cell suspension was stained with fluorescence-conjugated antibodies for 30 minutes at 4° C., and the stained cells were lysed in RLT lysis buffer (Qiagen, Valencia, Calif) and then used for RNA extraction through BD FACSAria II (BD Bioscience). The anti-human antibodies were purchased from Biolegend (Percp-Cy5.5-conjugated anti-CD8 [SK1]) and eBioscience (eF450-conjugated anti-human CD3 [OKT3], PE-conjugated anti-CD4 [OKT4], and APC-conjugated anti-CD69 [FN50]). T_{MM} cells: CD3⁺CD4⁺CD69⁻ or CD3⁺CD8⁺CD69⁻, T_{RM} cells: CD3⁺CD4⁺CD69⁺ or CD3⁺CD8⁺CD69⁺.

Total RNA was extracted using the RNeasy Micro Kit (Qiagen) according to the manufacturer's instructions, and immediately stored at −80° C. RNA purity and integrity were assessed using an ND-1000 spectrophotometer (NanoDrop, Wilmington, DE, USA).

Sequencing was performed on the extracted total mRNA (Macrogen Co., Ltd., Seoul), and a heat map was created using the R program (Package:ggplot2) based on the sequencing results (https://healthstat.snu.ac.kr/CRAN/).

### Toxicity Assay Using CCK-8 Reagent

To determine the final concentration of the drug, cells isolated from normal skin tissue were seeded in a 96-well plate at a density of 2×4⁵ cells per well using 5% (v/v) RPMI1640 medium (LONZA) containing 1% penicillin/streptomycin, and the cells were treated with idelalisib at varying concentrations of 1 μM, 10 μM, 50 μM, 100 μM, and 200 μM, and then incubated in an incubator at 37° C. overnight. Thereafter, 10 μL of Cell Counting Kit-8 reagent (Dojindo Lot. PR863) was added to each well of the 96-well plate, and after 2 hours, the safety and toxicity of the drug to the normal skin tissue were evaluated by measuring the absorbance at 540 nm.

### Measurement of Changes in Skin-Specific T Cells in NC/Nga Mice

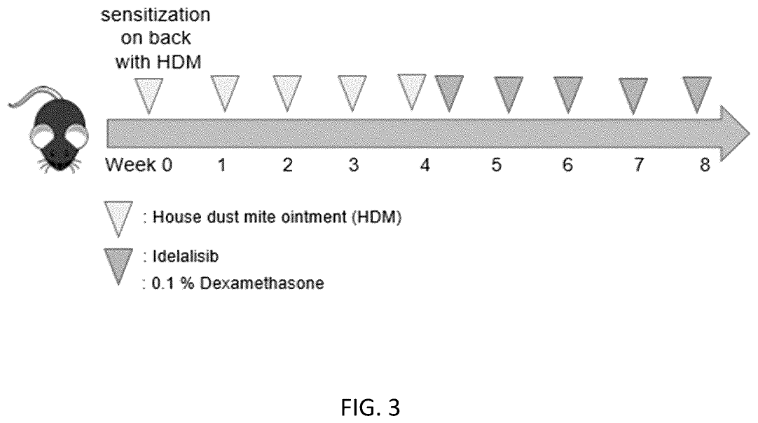

Atopic skin lesions were induced in NC/Nga mice by treatment with HDM three times a week until week 4 after the start of the experiment, and then three of the mice were each treated with 50 μM or 100 μM idelalisib (200 μL) and/or 0.1% dexamethasone (200 μL) three times a week for another four weeks (FIG. 3). Control groups consisted of a non-HDM-treated group (3 mice), a HDM-treated group (3 mice), and a HDM- and 0.1% dexamethasone-treated group (3 mice, positive control group), and experimental groups were administered idelalisib after HDM treatment. Then, in order to check changes in skin-specific T cells in each animal, single suspensions were prepared using the spleen, skin, and lymph nodes, and T-cells were stained using anti-CD3, anti-CD4, anti-CD8, and anti-CD45 antibodies. Flow cytometry was performed on skin-specific T cells using anti-CD69 and anti-CD103 antibodies, thereby observing the changes in number and distribution of skin-specific T cells by the application of the drug.

### ELISA

Blood was collected from the mouse heart, and then serum total DF-specific IgE (ng/mL) (A₄₅₀) was measured in a 96-well plate (Corning Inc., Corning, NY, USA) using the ELISA MAX™ Delux Set (BioLegend, San Diego, CA, USA) kit.

Blood was collected from the mouse heart and then centrifuged at 2,500 rpm for 15 minutes, and the obtained serum was used in the experiment. Then, total IgE was measured using the ELISA MAX™ Delux Set (BioLegend, San Diego, CA, USA) kit. Dermatophagoides farinae (DF)-specific IgE was measured by ELISA using a slight modification of the previously reported method. Briefly, 100 μL of 20 μg/mL DfE in 50 mmol/L carbonate buffer (pH 9.6, Sigma, St. Louis, MO, USA) per well was plated in a 96-well microtiter plate (Corning Inc., Corning)., NY, USA) and incubated overnight at 4° C. Each well was blocked with 3% skim milk in PBS (PBST) containing 0.05% Tween 20 for 1 hour at 37° C. After washing three times with PBST, each well was incubated with a serum sample (diluted to 1:20 [V/V] in 1% BSA) overnight at 4° C. After washing again three times with PBST, each well was incubated with biotinylated rat anti-mouse IgE at room temperature for 2 hours, and incubated with horseradish peroxidase (HRP)-streptavidin (Vector Laboratories, Burlingame, CA, USA) at room temperature for 1 hour. The color development reaction was initiated by adding 3,3′5,5′-tetramethylbenzidine solution (TMB solution, KPL Inc., Gaithersburg, MD, USA) to each well and stopped by adding TMB stop solution (KPL Inc.). The absorbance was measured at 450 nm with a microplate reader (TECAN, Salzburg, Austria).

Examination of Changes in Skin-Specific T Cells in Human Skin with Atopic Dermatitis Lesions

To verify the effect of the candidate drug on skin-specific T cells in human skin tissue, a single suspension was prepared using the skin from an atopic dermatitis patient. T cells were stained using anti-CD3, anti-CD4, anti-CD8 and anti-CD45 antibodies, and flow cytometry was performed on skin-specific T cells using anti-CD69 and anti-CD103 antibodies, which are skin T_{RM} markers, thereby observing the changes in number and distribution of skin-specific T cells by the application of the drug.

### Results

### Cytotoxicity in Normal Tissue

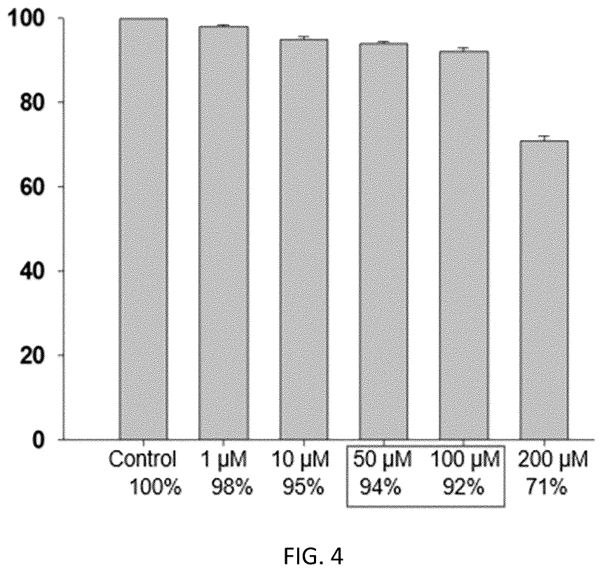

As a result of measuring the cell viability in normal skin tissue following drug administration by the Cell Counting Kit-8 reagent (Dojindo Lot. PR863), it was confirmed that idelalisib showed cell viabilities of 90% or more even at concentrations of 50 μM and 100 μM, indicating that idelalisib had no significant toxicity in normal skin tissue (FIG. 4).

### Gene Expression Profiles of Skin-Specific T Cells

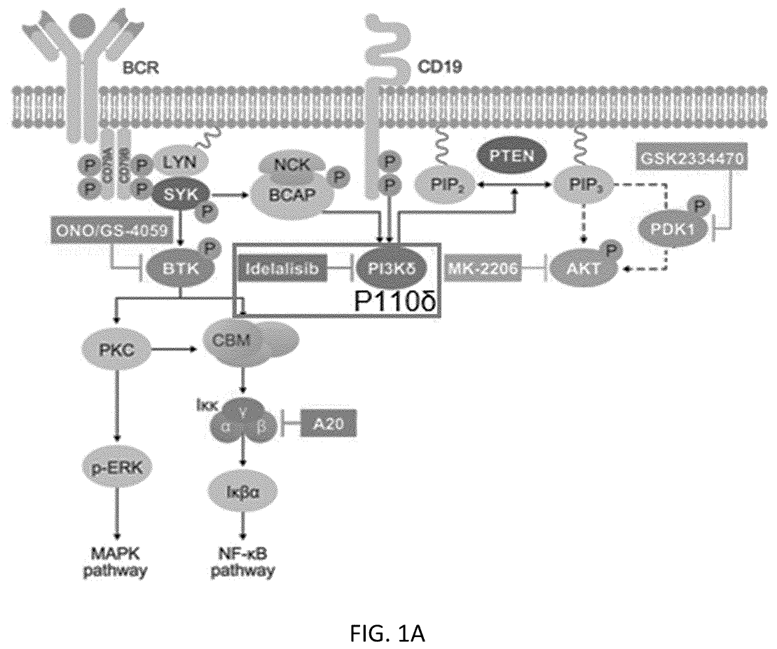

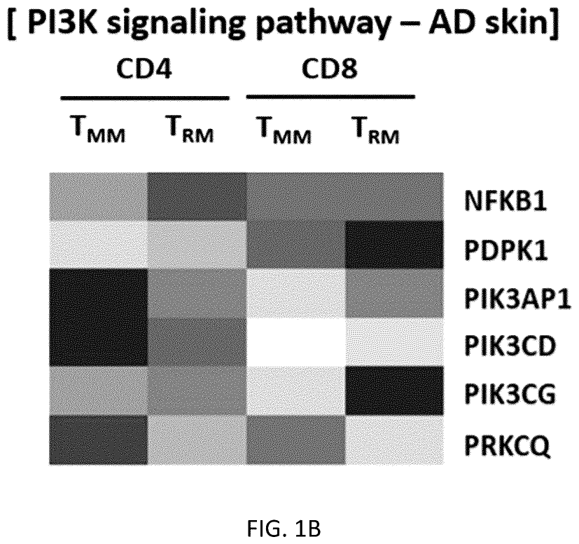

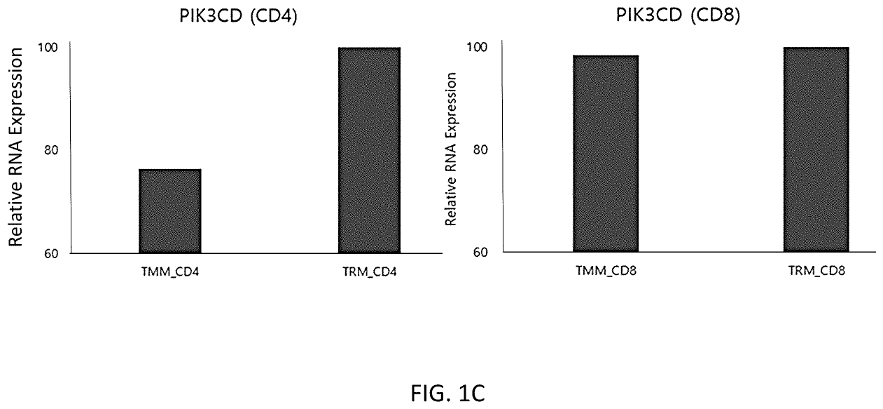

A heat map was created using R program the ((https://healthstat.snu.ac.kr/CRAN/). Next, PI3K-related genes were investigated by performing microarray analysis. First, the present inventors searched for genes whose expression level increased in CD4 T cells compared to CD8 T cells. Among these genes, PI3KCD was discovered as a gene that showed significantly increased expression in CD4 T_{MM} (migrated memory T cells) and T_{RM} (tissue-resident memory T cells) (FIG. 1C). Accordingly, PIK3CD was selected as a therapeutic target for atopic dermatitis.

### Comparison of Inflammatory Responses in Mice

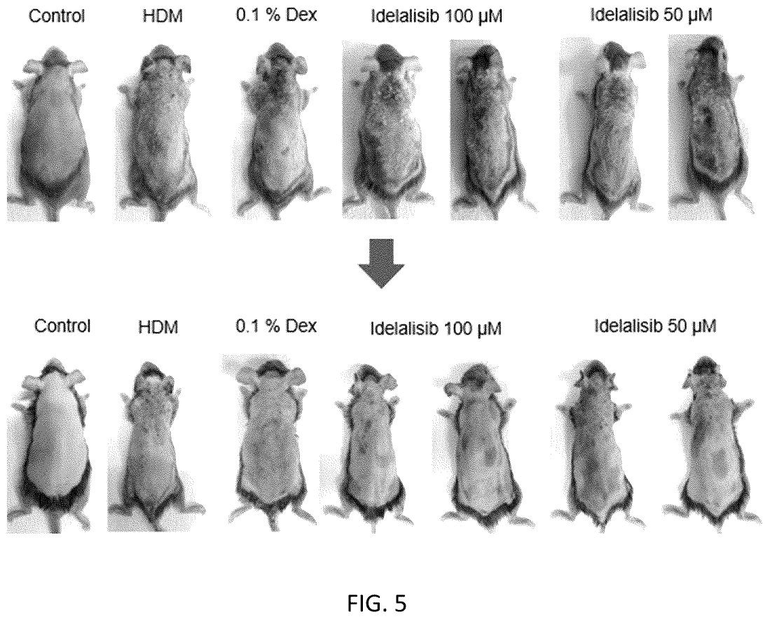

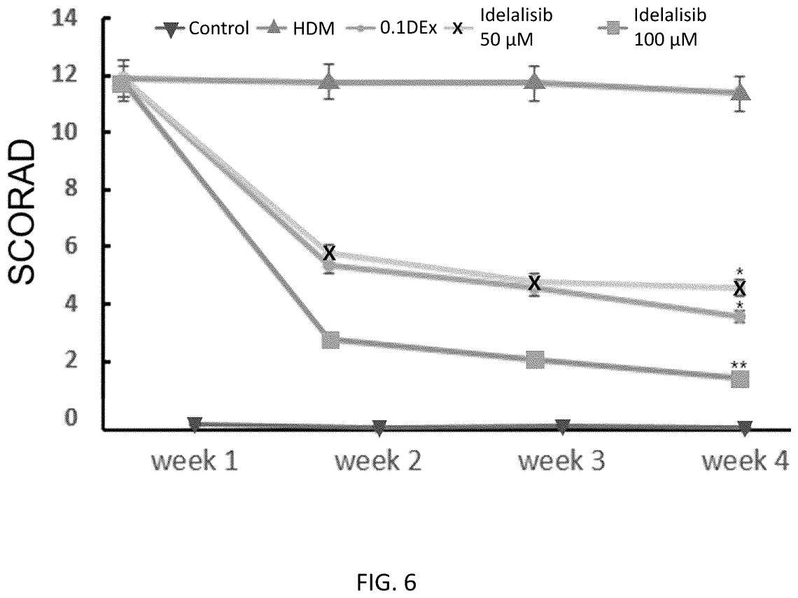

To evaluate the effect of idelalisib administration in NC/Nga mice with induced atopic dermatitis, atopic dermatitis was induced by applying house dust mite (HDM) ointment (Biostir, Osaka, Japan) to the mouse's back three times a week for 4 weeks, and then idelalisib drug was applied to the mouse's back three times a week for another 4 weeks. As a result, a significant therapeutic effect was observed in a drug concentration-dependent manner, and in particular, the most notable recovery was found in the group to which idelalisib was applied at a concentration of 100 μM (FIG. 5). In particular, erythema, bleeding, wounds, dryness, and shedding were significantly reduced even compared to the positive control group administered 0.1% dexamethasone. Accordingly, it could be confirmed that idelalisib exhibited clinically significant therapeutic effects in the atopic dermatitis mouse model. In addition, as a result of measuring the scoring atopic dermatitis (SCORAD) index of NC/Nga mice, the SCORAD index significantly decreased in the idelalisib-administered groups, and the idelalisib-administered groups showed a lower SCORAD index than the positive control group administered 0.1% dexamethasone, indicating that the idelalisib-administered groups showed better therapeutic responses against atopic dermatitis-like skin lesions such as erythema, bleeding, wounds, dryness, swelling, and shedding (FIG. 6). Each score was obtained by the Wilcoxon signed-rank test, and P<0.05 was considered statistically significant. Three mice per group were tested.

### Measurement of Serum Total IgE

The changes in serum total IgE levels in atopic dermatitis mouse models by idelalisib treatment were measured. In atopic dermatitis, when the skin is exposed to an allergen, antigen-specific IgE binds to the IgE receptor on the surface of Langerhans cells and is then transferred to T cells, thereby activating the T cells. Accordingly, the level of serum total IgE is known to tend to be high in patients with atopic dermatitis and to increase with the onset and worsening of the disease, indicating that it is a measure for diagnosis and severity assessment of atopic dermatitis.

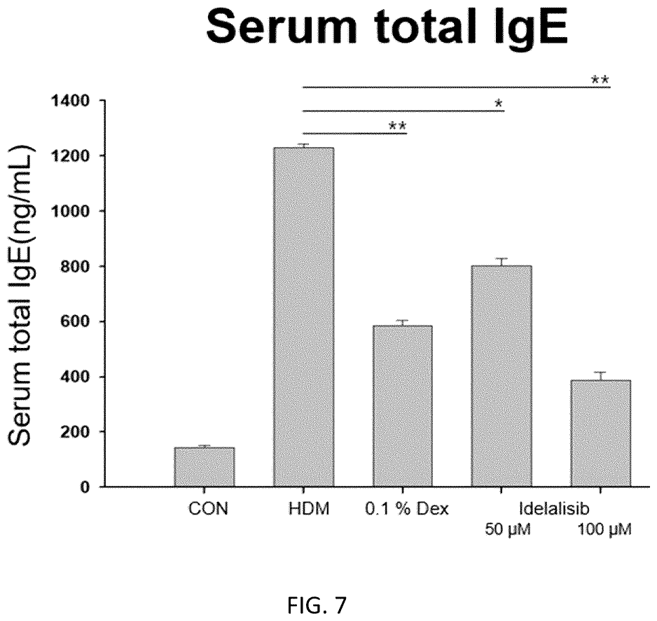

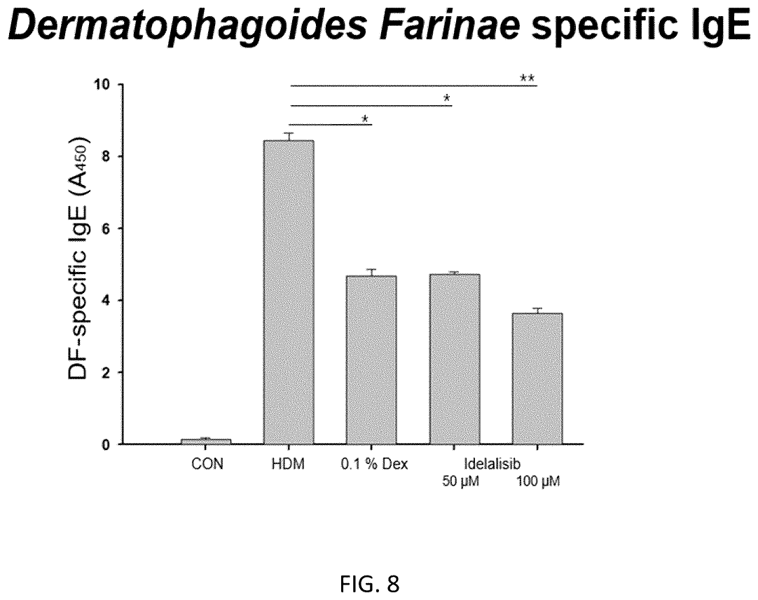

As a result of measuring total IgE using ELISA, it was confirmed that idelalisib treatment decreased the total IgE level in a dose-dependent manner. In particular, the decrease in the serum total IgE level was found to be significantly greater in the 100 μM idelalisib-treated group than in the 0.1% dexamethasone-administered group, which was used as a positive control group (FIG. 7).

### Measurement of Serum Total DF-Specific IgE

The sensitization rate to house dust mites (Dermatophagoides farinae, DF) in patients with atopic dermatitis has been reported to be 27.9% to 66.7%. Based on this fact, as a result of measuring total DF-IgE in NC/Nga mice sensitized to atopic dermatitis, it was confirmed that the drug idelalisib reduced the level of total DF-specific IgE in a dose-dependent manner. In particular, it was observed that the total DF-specific IgE level in the 100 μM idelalisib-treated group significantly decreased even compared to that in the 0.1% dexamethasone group, which was a positive control group, and the 50 μM idelalisib group also showed a decrease in the total DF-specific IgE level, which was similar to that in the 0.1% dexamethasone group (FIG. 8).

### H&E Staining

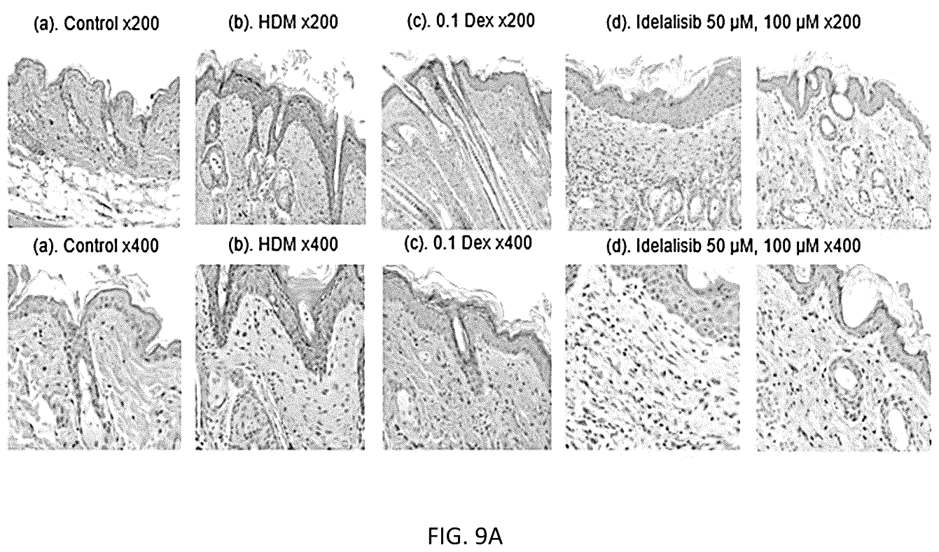

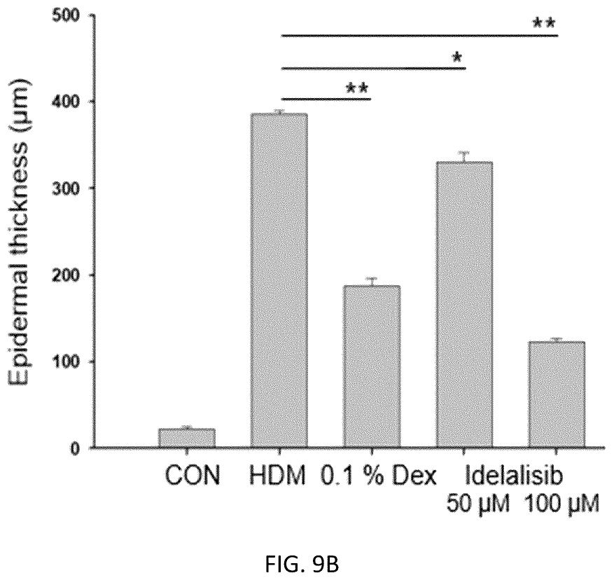

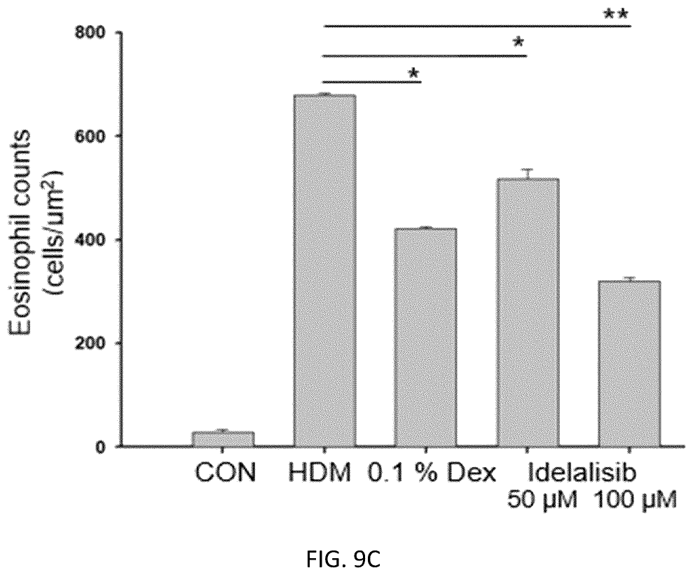

In atopic dermatitis, the eosinophil count is observed to be characteristically high, making it a representative biological indicator that can explain the immunological mechanism of atopic dermatitis. Based on this fact, hematoxylin and eosin (H&E) staining in NC/Nga mice was performed. As a result, it was confirmed that the eosinophil count decreased depending on the idelalisib concentration (FIG. 9C). In addition, when atopic dermatitis occurs, the skin thickens due to an excessive inflammatory response. It was confirmed that the epidermal thickness of the mice decreased as the concentration of idelalisib increased (FIG. 9B).

### FACS

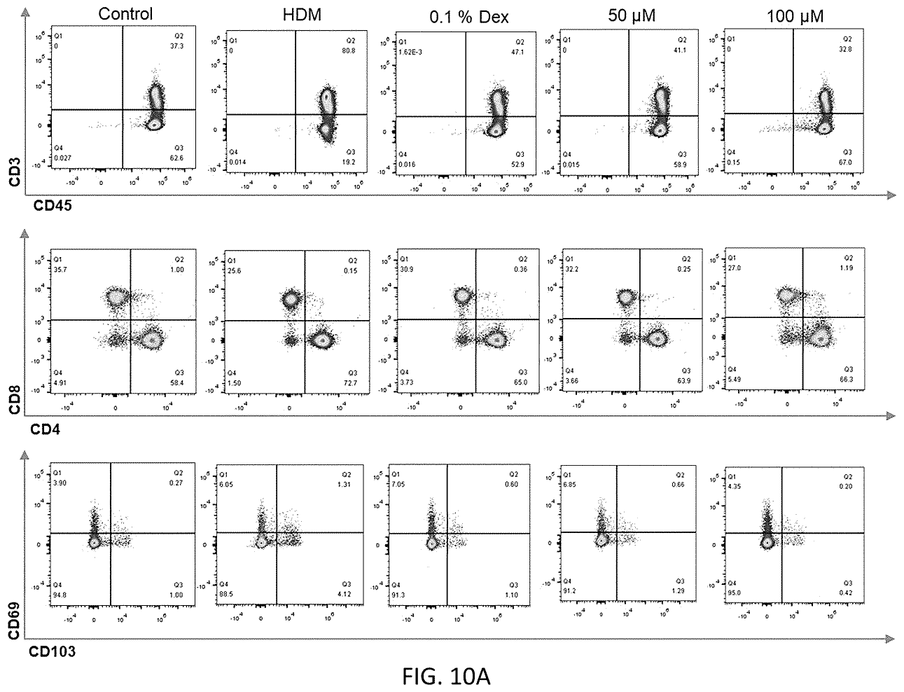

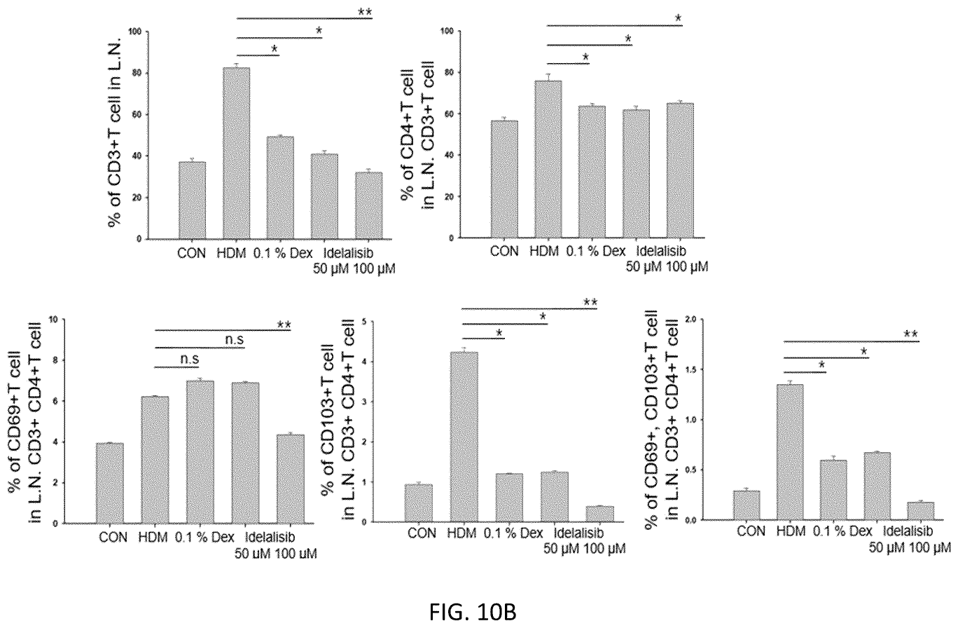

As a result of observing CD3+ T cells in skin-draining lymph nodes by FACS, the 100 μM idelalisib-treated group showed the lowest proportion of CD3+ T cells, which was statistically significantly (FIG. 10A). In addition, it was confirmed that the fraction of T cells expressing CD69 among CD3+ CD4+ T cells also tended to decrease, and the fraction of CD3+ CD4+ CD103+ T cells was also statistically significantly lowest in the 100 μM idelalisib-treated group (FIG. 10B), suggesting that the numbers of CD69+ T cells and CD103+ T cells, which are known to increase in number in atopic dermatitis skin lesions, were statistically significantly reduced.

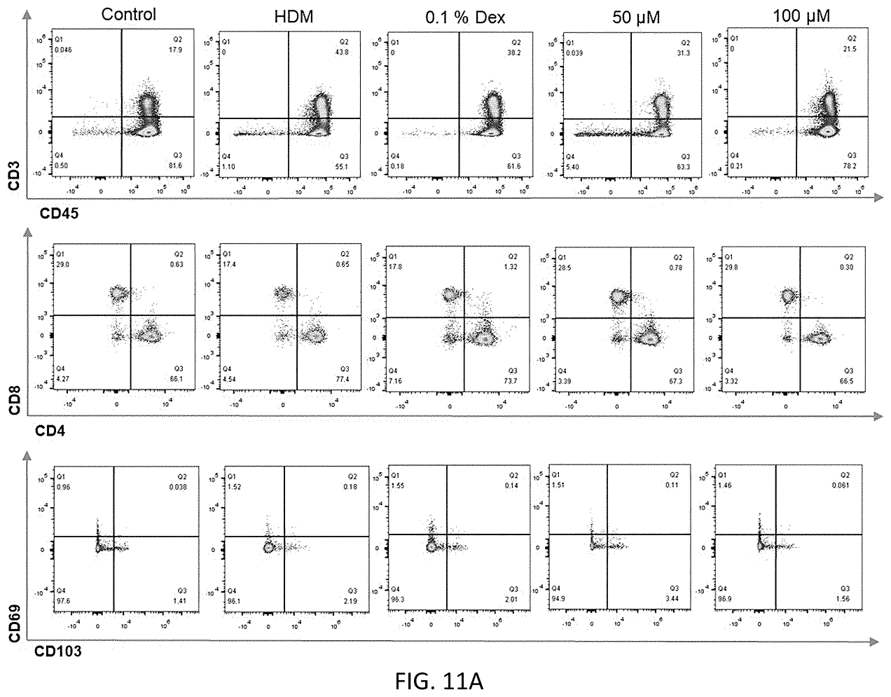

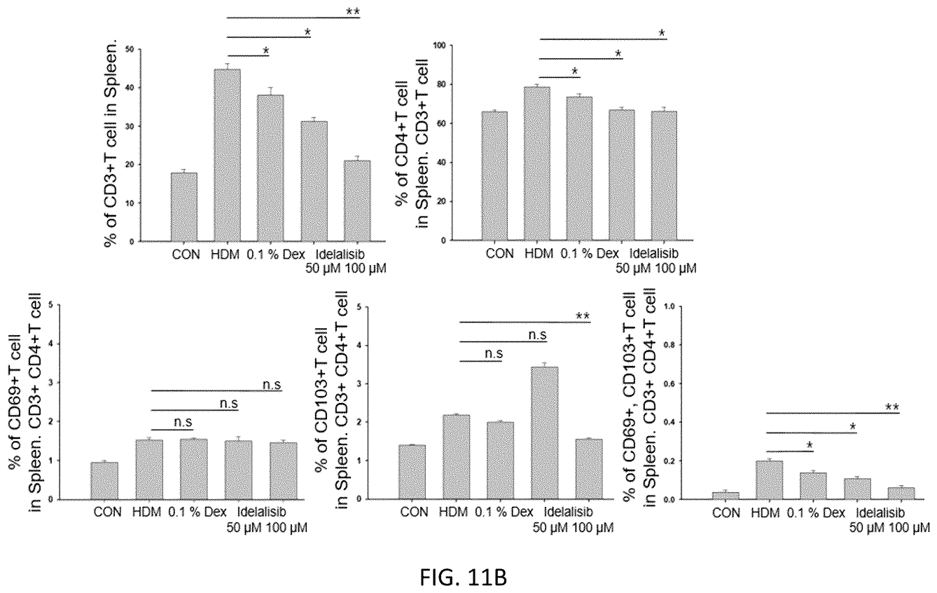

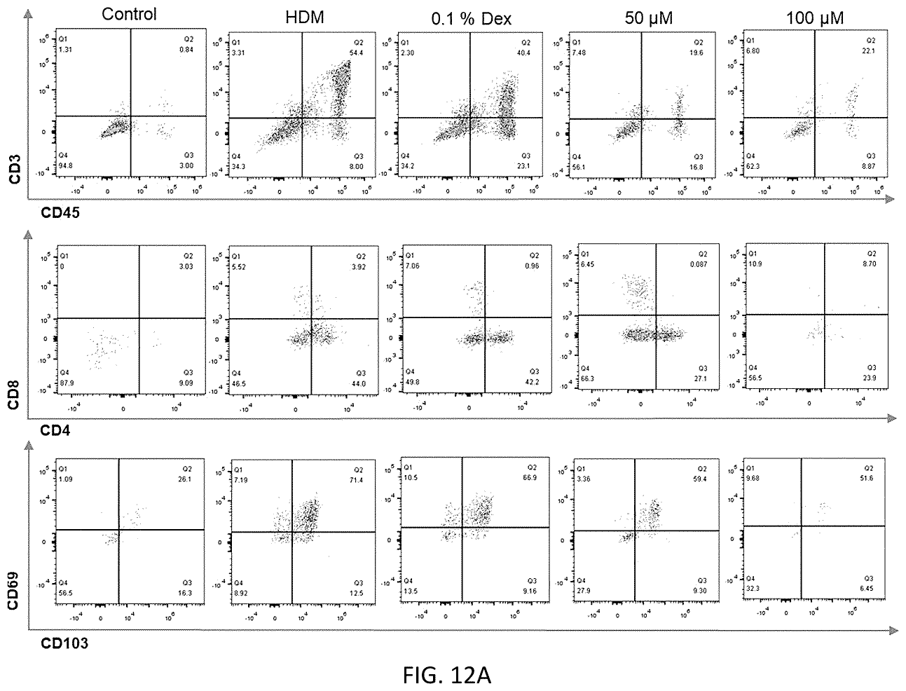

In addition, it was confirmed that, the proportion of CD3+ and CD4+ T cells in the spleen decreased in a dose-dependent manner by idelalisib treatment, and in particular, CD69+ T cells and CD103+ T cells, known as T_{RM} markers, also decreased statistically significantly in the spleen (FIG. 11). Furthermore, it was confirmed that CD69+ T cells and CD103+ T cells significantly decreased in the idelalisib-treated group compared to the 0.1% dexamethasone group, which was a positive control group.

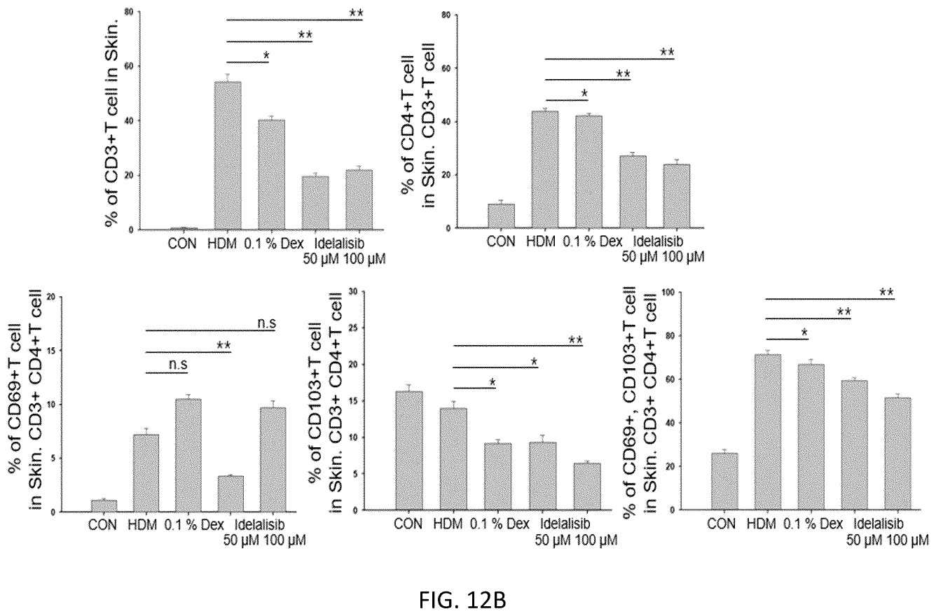

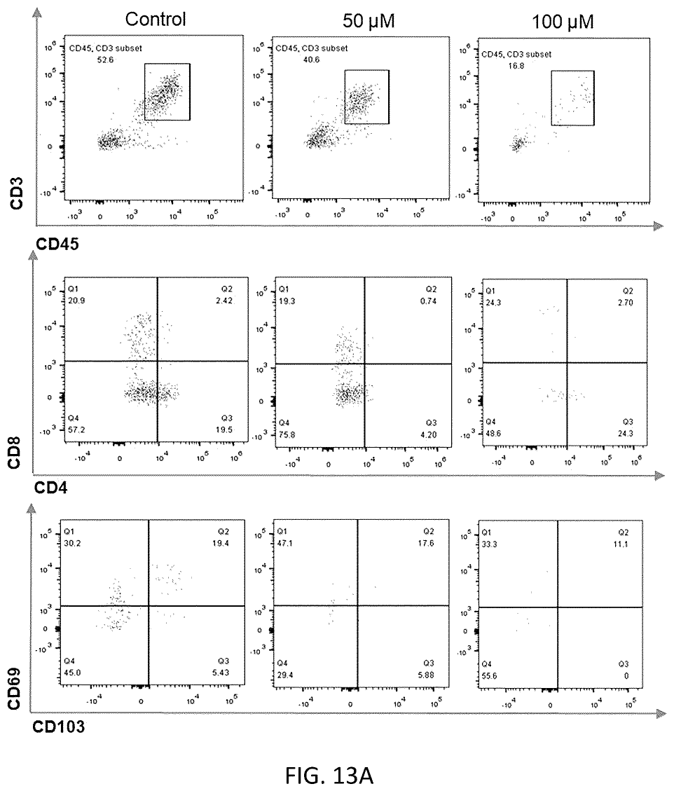

In addition, it was confirmed that the proportions of CD3+ and CD4+ skin-specific T cells in the skin significantly decreased in both the 50 μM and 100 μM idelalisib groups in a dose-dependent manner. In particular, it was confirmed that CD69+ T cells and CD103+ T cells also statistically significantly decreased in the skin tissues of the idelalisib groups, and that the proportions of skin-specific T cells in the idelalisib groups significantly decreased even compared to that in the 0.1% dexamethasone-treated group, which was a positive control group (FIG. 12).

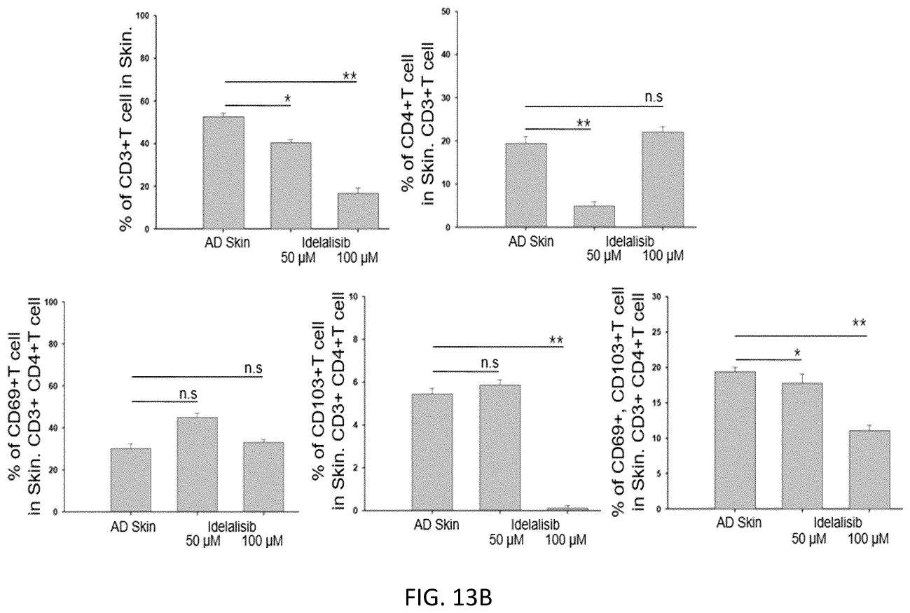

Finally, as a result of analyzing T cells by FACS using skin immune cells and skin lesions of atopic dermatitis patients, it was confirmed that the proportion of skin-specific T cells significantly decreased in both the 50 μM and 100 μM idelalisib groups in a dose-dependent manner (FIG. 13).

Based on the above results, it can be found that the composition of the present invention effectively treats the symptoms of atopic dermatitis through a multifaceted mechanism, and controls immune and inflammatory responses in a skin tissue-specific manner, especially by inhibiting skin-specific T cells.

Although the present invention has been described in detail with reference to the specific features, it will be apparent to those skilled in the art that this description is only of a preferred embodiment thereof, and does not limit the scope of the present invention. Thus, the substantial scope of the present invention will be defined by the appended claims and equivalents thereto.

1-12. (canceled)

13. A method for preventing or treating an inflammatory or autoimmune skin disease, the method comprising administering a composition comprising a compound of Formula 1 or a pharmaceutically acceptable salt thereof as an active ingredient to a subject in need thereof: 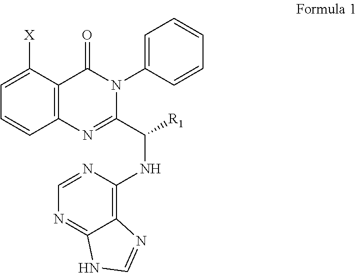
wherein X is halogen, and R₁ is C₁-C₃ alkyl.

14. The method according to claim 13, wherein X is F.

15. The method according to claim 13, wherein R₁ is C₂ alkyl.

16. The method according to claim 13, wherein the inflammatory or autoimmune skin disease is selected from the group consisting of atopic dermatitis, eczema, erythema multiforme, erythema nodosum, and pyoderma gangrenosum.

17. The method according to claim 16, wherein the inflammatory or autoimmune skin disease is atopic dermatitis.

18. The method according to claim 13, wherein the composition reduces a number or activity of skin-specific T cells.

19. The method according to claim 13, wherein the composition is a composition for transdermal administration or topical skin application.

20. A method for alleviating or ameliorating an inflammatory or autoimmune skin disease, the method comprising administering a cosmetic composition comprising a compound of Formula 1 or a cosmetically acceptable salt thereof as an active ingredient to a subject in need thereof: 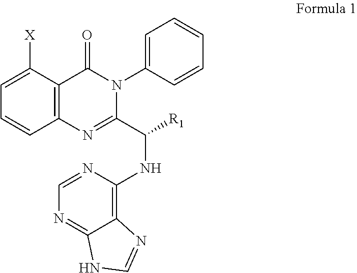
wherein X is halogen, and R₁ is C₁-C₃ alkyl.

21. The method according to claim 20, wherein the inflammatory or autoimmune skin disease is atopic dermatitis.

22. The method according to claim 20, wherein the cosmetic composition reduces a number or activity of skin-specific T cells.

23. A method for screening a composition for inhibiting skin-specific T cells, the method comprising the steps of:
(a) bringing a candidate substance into contact with a biological sample containing cells expressing PIK3CD (phosphatidylinositol-4,5-bisphosphate 3-kinase catalytic subunit delta) protein; and
(b) measuring an activity or expression level of the PIK3CD protein in the biological sample,
wherein, if the activity or expression level of the PIK3CD protein decreases, the candidate substance is determined as the composition for inhibiting skin-specific T cells.

24. The method according to claim 23, wherein the biological sample contains skin tissue or skin tissue-derived cells.
